(19) United States
(12) Patent Application Publication
(10) Pub. No.: US 2024/0238319 A1
(43) Pub. Date: Jul. 18, 2024

(54) CEREAL-BASED COMPOSITIONS WITH A MIX OF GALACTO-OLIGOSACCHARIDES/FRUCTO- OLIGOSACCHARIDES AND USES THEREOF FOR IMPROVING IRON ABSORPTION

(71) Applicant: COMPAGNIE GERVAIS DANONE, Paris (FR)

(72) Inventors: Raphaëlle BOURDET-SICARD, Palaiseau (FR); Morgane BERGEONNEAU, Lyon (FR); Michaël Bruce ZIMMERMANN, Rapperswil-Jona (CH)

(21) Appl. No.: 18/564,495

(22) PCT Filed: May 27, 2021
(86) PCT No.: PCT/IB2021/000379

§ 371 Date: Nov. 27, 2023

(51) Int. Cl. A61K 31/702; A23L 7/10; A61K 9/00; A61K 33/26; A61P 7/06

(52) U.S. Cl. CPC A61K 31/702; A23L 7/101; A61K 9/0056; A61K 33/26; A61P 7/06

(57) ABSTRACT

The present invention is in the field of cereal-based compositions for preventing and/or treating iron deficiency, in particular intended for infants, young children, women of reproductive age including pregnant or lactating women, and elderly. The invention relates to a composition comprising at least a mixture of galacto-oligosaccharides (GOS) and fructo-oligosaccharides (FOS), cereal and iron and uses thereof.

### FIELD OF THE INVENTION

The present invention is in the field of cereal-based compositions for preventing and/or treating iron deficiency, in particular intended for infants, young children, women of reproductive age including pregnant or lactating women, and elderly.

### TECHNICAL BACKGROUND

Fractional iron absorption (FIA) from iron-fortified foods or oral iron supplements is generally low; often less than 10% of the iron dose is absorbed. Thus, the majority of iron passes unabsorbed into the colon where it favors growth of potential enteropathogens, for which iron is crucial for replication and virulence, over important commensal ‘barrier’ strains such as bifidobacteria and lactobacilli, which require little or no iron. Studies conducted in African infants and children have shown that in settings with poor hygiene and a high burden of infection and inflammation, iron fortification and supplementation adversely affect the gut microbiota, decrease beneficial bifidobacteria and lactobacilli, increase enteropathogens and increase gut inflammation. These adverse changes in the gut microbiota provide a plausible mechanism for the reported increased risk of diarrhea with provision of iron to infants and children in low-resource settings. A recent systematic review reported a 15% increased risk of diarrhea with iron when providing at least 80% of the WHO recommended dietary allowance.

Promising strategies to reduce the adverse effects of iron on the infant gut is to use the lowest possible dose of iron with proven efficacy and the co-provision of prebiotic fibers. As non-digestible carbohydrates, prebiotics enter the colon intact, where they can selectively enhance the growth of beneficial commensal bifidobacteria and lactobacilli. Prebiotics may protect from colonization and overgrowth of potential enteric pathogens by increasing colonization resistance, increasing production of short chain fatty acids and decreasing intestinal luminal pH. A 4-month trial in Kenyan infants showed that the addition of galacto-oligosaccharides (GOS) to a low-dose iron micronutrient powder (MNP) (5 mg iron) mitigated most of the adverse effects of iron on the infant gut microbiota (Paganini et al., Gut., 66(11):1956-67, 2017).

Prebiotics may also increase iron absorption from fortified foods and MNPs (Paganini et al., The American Journal of Clinical Nutrition 2017:1020-31, 2017). In iron deficient Kenyan infants who consumed a MNP containing 5 mg of a mixture of ferrous fumarate (FeFum) and sodium iron EDTA (NaFeEDTA) and 7.5 g GOS or the same MNP without GOS daily for three weeks, GOS consumption significantly increased iron absorption by 62% from FeFum+NaFeEDTA, but did not increase iron absorption significantly from FeSO₄. However, this study could not distinguish whether the effect of GOS on iron absorption was an acute effect (i.e. from addition to the single test meal) or a chronic effect (i.e. prior consumption of GOS daily for several weeks), or a combination of these effects. In iron-deplete women, iron absorption from FeFum in a test meal and in water before (baseline) and after daily consumption of GOS for four weeks was measured (Jeroense et al., The Journal of Nutrition, 149(5):738-46, 2019). At baseline, GOS significantly increased iron absorption from FeFum when given with water (+61%) and the meal (+28%), and after four weeks of GOS consumption, GOS again significantly increased absorption from FeFum in the meal (+29%). However, compared with baseline, consumption of GOS for four weeks did not significantly enhance absorption from FeFum in the meal given without GOS. More recently, another study showed that the addition of GOS to a single iron-fortified maize porridge test meal in Kenyan infants had no acute effect and did not significantly increase iron absorption (Mikulic et al., The Journal of Nutrition, 151(5):1205-1212, 2021).

Thus, the effects of GOS on iron absorption are still uncertain and there remains a genuine need for new compositions capable of providing a low dose of iron with high bioavailability. The present invention is believed to meet such need.

### SUMMARY OF THE INVENTION

Unexpectedly, the Inventors have found that the addition of a mixture of galacto-oligosaccharides (GOS) and fructo-oligosaccharides (FOS) in a cereal-based composition comprising iron results in a significant increase in FIA. Moreover, they observed that the addition of GOS+FOS results in a significant increase in both “acute” (i.e. obtained by a single dose of GOS+FOS) and “chronic” (i.e. obtained by the consumption of GOS+FOS daily for three weeks) FIA from the cereal-based composition. It was also observed a surprising “conditioning” effect of the mixture of GOS+FOS on FIA (i.e. after the consumption of GOS daily for three weeks, the FIA remains increased for a meal without GOS+FOS).

The present invention thus concerns compositions comprising at least a mixture of GOS+FOS, cereal and iron, which can be used to treat and/or prevent iron deficiency or anemia. These compositions are particularly advantageous since they increase bioavailability of iron with no need to wait weeks to have iron absorption improvement and they allow to reduce the amount of iron consumed by a subject, thus limiting the adverse effects of iron on the gut microbiota. Furthermore, due to their conditioning effect, these compositions are also particularly useful since their consumption can even improve the iron absorption from a meal without GOS+FOS.

### DETAILED DESCRIPTION OF THE INVENTION

### Composition

The invention relates to a composition comprising at least a mixture of galacto-oligosaccharides (GOS) and fructo-oligosaccharides (FOS), cereal and iron.

In an embodiment, the composition of the invention can be a dry composition. Such a dry composition can be reconstituted with a food grade liquid to obtain a ready to feed composition.

In an embodiment, the composition of the invention further comprises milk powder.

In an embodiment, the composition of the invention comprises a food grade liquid, preferably selected from the group consisting of water, follow on formula or milk (from animal or vegetal origin).

The composition of the invention has improved bio-availability of iron and can increase the absorption of iron by a subject. The composition according to embodiments of the invention may thus be a therapeutic composition, such as a medicament or a pharmaceutical composition, or a non-therapeutic composition, such as a nutritional composition, a nutraceutical composition, a nutritional supplement and/or a food composition.

For instance, the nutritional composition according to the invention include, but are not limited, to a nutritional formula, an infant formula, a baby food, a baby food puree, a weaning food, a cereal porridge, an infant follow-on formula, a young child formula and/or a formula for women of reproductive age including pregnant or lactating women and/or elderly. These subjects, weaning infants, children, women of reproductive age and elderly have a higher risk of suffering from iron deficiency and, thus, they benefit the most from the composition of the invention. In the context of the invention, the compositions can provide partial or complete nutritional requirements of a subject.

Therapeutic or non-therapeutic compositions of the invention may contain further ingredients. The exact nature and ratio of these further ingredients may differ depending on the type of composition. The skilled person is well aware that the nutritional requirements of a weaning infant nutrition differ from those of a supplement for lactating women, and is able to adjust the composition accordingly. For example, the composition of the invention may contain further ingredients, such as proteins, lipids, further carbohydrates (digestible and non-digestible), vitamins (in particular A, B1, B3, B5, B6, B8, B9, C, D3, E), minerals, salt, emulsifiers and flavor agents.

The present invention relates to compositions, as well as several uses or applications of the compositions. Hence, all defined herein for the compositions according to the invention equally applies to the uses and applications according to the invention, and vice versa.

### GOS/FOS

The compositions of the invention comprise a mixture of galacto-oligosaccharides (GOS) and fructo-oligosaccharides (FOS). GOS and FOS are non-digestible oligosaccharides. They are not digested in the intestine by the action of enzymes present in the human upper digestive tract (small intestine and stomach) but they are fermented by the human intestinal microbiota.

As used herein, “galacto-oligosaccharides” (or “GOS”) may refer to β-galacto-oligosaccharides, α-galacto-oligosaccharides and galactan. Preferably, GOS are β-galacto-oligosaccharides. Preferably, GOS comprise galacto-oligosaccharides with β(1,4), β(1,3) and/or β(1,6) glycosidic bonds and a terminal glucose. For example, GOS suitable for use in the present invention are commercially available under the trade name VIVINAL®GOS, VINAL®GOS (Friesland Campina Ingredients), Bi2muno (Clasado), Cup-oligo (Nissin Sugar) and Oligomate55 (Yakult).

As used herein, “fructo-oligosaccharides” (or “FOS”) may refer to oligosaccharides comprising β-linked fructose units, which are preferably linked by β(2,1) and/or β(2,6) glycosidic linkages. Preferably, FOS contain a terminal β(2,1) glycosidic linked glucose. Preferably, FOS contain at least 7 β-linked fructose units. In a preferred embodiment, inulin is used. Inulin is a type of FOS wherein at least 75% of the glycosidic linkages are β(2,1) linkages. For example, FOS suitable for use in the present invention are commercially available under the trade name Orafti®HP and Orafti®HPX (Beneo), FIBRULOSE® and FIBRULINE® (Cosucra) and FRUTAFIT® and FRUTALOSE® (Sensus).

In the present invention, both GOS and FOS are present in the compositions. They are referred to as a mixture of GOS and FOS (“GOS+FOS”, “GOS/FOS” or “GOS:FOS”).

Advantageously, GOS and FOS are water-soluble (according to the method disclosed in L. Prosky et al., J. Assoc. Anal. Chem 71:1017-1023, 1988).

They have preferably a degree of polymerisation (DP) of 3 to 200. The average DP of GOS and FOS is preferably below 200, more preferably below 100, even more preferably below 60.

Preferably, GOS are short chain GOS (scGOs), i.e. they have a degree of polymerization (DP) from 3 to 10. Preferably, FOS are long chain FOS (lcFOs), i.e. they have a DP from 5 to 60. In a most preferred embodiment, the mixture of GOS+FOS is a mixture of scGOS and lcFOS. Preferably, the mixture of GOS/FOS comprises scGOS with an average DP below 10, preferably below 6, and lcFOS with an average DP above 7, preferably above 11, even more preferably above 20.

Preferably, the mixture of GOS and FOS is present in a weight ratio of from 20:1 to 1:20, more preferably from 10:1 to 1:10, even more preferably from 9:1 to 1:9. In the most preferred embodiment, the mixture of GOS+FOS is in a weight ratio of 9:1. These weight ratios are particularly advantageous when the GOS are scGOS and the FOS are lcFOS.

The compositions of the invention preferably comprise from 1 to 30% GOS+FOS, more preferably from 1 to 20%, even more preferably from 2 to 10%, based on dry weight of the total product. In some embodiments, the compositions of the invention comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30% GOS+FOS based on dry weight of the total product.

### Cereals

Cereals are food products that are particularly appropriate to be used in the weaning period, in healthy infants and young children aged six months or older. When fortified in iron, they can contribute significantly to iron intake, bringing 30-60% of the Reference Nutrient Intakes (RNIs) of iron, being 9.3 mg per day for 6-12 month old children, assuming 10% absorption rate, in order to decrease the risk of Iron Deficiency Anemia (IDA).

Any cereal can be used in the context of the present invention. In some embodiments, the cereal is selected from the group consisting of wheat, millet, sorghum, rice, teff, rye, spelt, barley, oat, soy, maize, semolina, buckwheat, tapioca, quinoa, amaranth, brown rice, wild rice, bulgur, farro, freekeh, khorasan wheat and combinations thereof, most preferably the cereal is wheat. The cereal may be a mixture of cereals, wherein it is preferred that at least one is selected from the list above.

In an embodiment, the cereal is in the form of flakes, flour or powder.

In an embodiment, the cereal is non-fermented. In an embodiment, the cereal is not selected from the group of fermented cereals. In an embodiment, the composition does not contain lactic acid bacteria. In an embodiment, the composition does not contain components that could result from, or could be produced during, a fermentation step with lactic acid bacteria such as bacterial cell fragments, bioactive compounds or lactic acid.

The compositions of the inventions preferably comprise at least 5%, more preferably at least 10%, more preferably at least 25% of cereal based on dry weight of the total product. In some embodiments, the compositions of the invention comprise from 5 to 50%, preferably from 10 to 50%, more preferably from 25% to 50% of cereal based on dry weight of the total product. In some embodiments, the compositions of the invention comprise about 5, 10, 15, 20, 25, 30, 35, 40, 45 or 50% of cereal based on dry weight of the total product.

Iron Any iron source can be used in the context of the present invention. In the context of the invention, iron means Fe²⁺ or Fe³⁺. In some embodiments, the iron can be selected from the group consisting of ferrous fumarate, ferrous sulphate, ferrous lactate, ferrous gluconate, ferrous bisglycinate, ferrous citrate, ferric diphosphate, ferric pyrophosphate, ferric ammonium citrate and mixtures thereof, preferably ferrous fumarate (FeFum). Wherever in this description an amount or concentration of iron is mentioned, this refers to the amount or concentration of Fe²⁺ or Fe³⁺, hence excluding the weight of the counter ion such as fumarate, sulphate, lactate, of the iron source. Sources of ferrous iron are preferred as sources of ferric iron need to be converted to ferrous iron in the body, the capacity of which may be limited in human subjects with an age of 0 to 36 months, e.g. infants and young children. In an embodiment, the iron is mainly ferrous fumarate. This source of iron has a higher relative bioavailability of iron compared to ferric diphosphate.

The iron is preferably in combination with ascorbic acid (AA orvitamin C). It is recommended that ascorbic acid be present at a molar ratio of more than 1:2 (Fe:AA), preferably at a molar ratio of 1:3 (Fe:AA). In a preferred embodiment, the iron is ferrous fumarate and it is in combination with AA at a FeFum:AA molar ratio of 1:3.

In the present invention, the amount of iron fortification is reduced while maintaining or increasing the iron bioavailability, because of the effect of the mixture of GOS+FOS. Such reduced amount of iron fortification has beneficial effects on the intestinal microbiota and intestinal physiology of the consumer. Also, a reduced amount of fortified iron has beneficial effects for the product itself, for which the shelf-stability is further increased with respect to cereal compositions comprising a greater amount of fortified iron.

The compositions of the invention preferably comprise at least 1 mg, preferably at least 3 mg iron per 100 g dry weight of the total product. The compositions of the invention preferably comprise not more than 100 mg iron per 100 g dry weight, more preferably not more than 50 mg iron per 100 g dry weight, even more preferably not more than 10 mg iron per 100 g dry weight. In some embodiments, the compositions of the invention comprise from 1 to 10 mg, more preferably from 3 to 7 mg iron per 100 g dry weight of the total product. In some embodiments, the compositions of the invention comprise about 1, 2, 3, 4, 5, 6, 7, 8, 9 or 10 mg iron per 100 g dry weight of the total product. The composition of the invention have improved iron bioavailability which allows slightly lower iron concentrations than typically present in nutritional compositions such as weaning foods.

### Applications

The compositions of the present invention are intended for consumption by a subject, typically to be drunk or eaten by human subjects. The compositions are typically consumed by subjects after reconstitution of a dry composition with a food grade liquid. Preferred target groups are defined here below. The compositions of the invention are particularly suitable to be administered to subjects, typically human subjects at risk to be affected by, or affected by, iron deficiency, such as infants, young children, women of reproductive age and elderly.

In an embodiment, the compositions of the invention are for use in providing nutrition to human subjects with an age of 0 to 36 months. Infants are defined as human subjects with an age of below 12 months. Young children, or toddlers, are defined as human subjects with an age of 12 to 36 months. So in other words, the compositions of the invention are suitable for human subjects with an age of 0 to 36 months.

Healthy full term infants are born with a supply of iron that usually lasts for 4 to 6 months. Preferably, the compositions are suitable for a human subject with an age of 4 months to 36 months. In an embodiment, the compositions are preferably for use in providing nutrition to a human subject with an age of 4 months to 36 months. These infants or young children have a higher need for iron and are therefore more prone to suffer from iron deficiency or anemia.

Preterm infants have less iron stores, which are built up in the third trimester of pregnancy. Preterm infants, defined as infants born before week 37 of gestation, preferably before week 32, are in particular at risk of iron deficiency or anemia. In a preferred embodiment, the compositions of the invention are suitable for a preterm infant, preferably for a preterm infant born before week 37 of gestation, more preferably for a preterm infant born before week 32 of gestation.

In an embodiment, the compositions of the invention are suitable for, or suitable for administration to, women of reproductive age including pregnant or lactating women. Pregnant or lactating women are in higher need for iron and are therefore more prone to suffer from iron deficiency or anemia.

In an embodiment, the compositions of the invention are suitable for, or suitable for administration to, elderly. As used herein, elderly typically refers to the group of humans having an age above 55 years, preferably above 65 years.

The compositions of the invention are preferably enterically administered, more preferably orally administered.

In some embodiments, the methods and uses defined herein are non-medical, in particular when the compositions of the invention are administered to a healthy subject.

In some embodiments, the methods and uses defined herein are medical, in particular when the compositions of the invention are administered to a subject with iron deficiency or suffering from anemia.

In some embodiments, the compositions of the invention are for use as a medicament.

Some embodiments relate to a composition of the invention for use in treating or preventing anemia and/or iron deficiency. In other words, some embodiments concern a method for preventing or treating anemia and/or iron deficiency, comprising administering a composition of the invention to a subject in need thereof. In other words, some embodiments concern the use of a composition of the invention in the manufacture of a medicament for treating or preventing anemia and/or iron deficiency.

Some embodiments relate to a composition of the invention for use in increasing iron absorption, iron bioaccessibility and/or iron bioavailability, more preferably iron bioavailability. In other words, some embodiments concern a method for increasing iron absorption, iron bioaccessibility and/or iron bioavailability, comprising administering a composition of the invention to a subject in need thereof. In other words, some embodiments concern the use of a composition of the invention in the manufacture of a medicament for increasing iron absorption, iron bioaccessibility and/or iron bioavailability.

Some embodiments relate to a composition of the invention for use in improving cognitive development, improving motor development and/or improving socio-emotional development in a human subject with an age of 0 to 36 months or for preventing cognitive disorders, motor disorders and/or socio-emotional disorders in a human subject with an age of 0 to 36 months. In other words, some embodiments concern a method for improving cognitive development, improving motor development and/or improving socio-emotional development or for preventing cognitive disorders, motor disorders and/or socio-emotional disorders comprising administering a composition of the invention to a human subject with an age of 0 to 36 months in need thereof.

In other words, some embodiments concern the use of a composition of the invention in the manufacture of a medicament for improving cognitive development, improving motor development and/or improving socio-emotional development in a human subject with an age of 0 to 36 months or for preventing cognitive disorders, motor disorders and/or socio-emotional disorders in a human subject with an age of 0 to 36 months.

As used herein, “bioaccessibility” of iron is the amount of ingested iron that is potentially available for absorption and is dependent on digestion and/or release from the food matrix.

As used herein, “bioavailability” of iron is the amount of ingested iron that is absorbed and available for physiological functions, and is dependent on digestion and/or release from the food matrix, absorption by intestinal cells and transport to the body cells. Absorption is the uptake of a nutrient into the cell and is dependent on digestion and/or release form the food matrix.

As used herein, “anemia” is a decrease in number of red blood cells or less than the normal quantity of hemoglobin in blood. In the present invention, anemia refers in particular to iron deficiency anemia, i.e. anemia caused by insufficient iron bioavailability. Iron-deficiency anemia is caused by insufficient dietary intake and absorption of iron and causes approximately half of all anemia cases in the world. According to the WHO anemia is defined as a hemoglobin content of less than 6.83 mmol/l blood (≈less than 110 g/l) in infants or young children of 6 months to 5 years, of less than 7.13 mmol/l (≈less than 115 g/l) in children of 5 to 11 years of age, of less than 7.45 mmol/l (≈less than 120 g/l) in teens of 12 to 14 years of age, of less than 7.45 mmol/l (≈less than 120 g/l) in non-pregnant women with age above 15 years, of less than 6.83 mmol/l (≈less than 110 g/l) in pregnant women, and of less than 8.07 mmol/l (z less than 130 g/l) in men above 15 years of age. Symptoms are pallor, fatigue, light-headedness and weakness. Other symptoms can be headaches, trouble sleeping, loss of appetite, paleness, reduced resistance to infection, fragile nails. Iron-deficiency anemia for infants in their earlier stages of development has greater consequences than it does for adults. An infant made severely iron-deficient during its earlier life cannot recover to normal iron levels even with iron therapy. Iron-deficiency anemia affects neurological development by decreasing learning ability, negatively altering motor functions and negatively effecting socioemotional functioning as behavior. Additionally, iron-deficiency anemia has a negative effect on physical growth and immunity. In pregnant women, of whom it is estimated that 50% suffer from iron deficiency or anemia in Africa or South-East Asia, there is an increased need for iron. Maternal anemia may increase the risk of preterm or small birth weight babies.

As used herein, “Iron deficiency” (sideropaenia or hypoferraemia) is a stage preceding iron deficiency anemia. The body has less than adequate iron stores. It can for example be determined by measuring an abnormal value for at least two of the three following indicators, serum ferritin, transferrin saturation, and free erythrocyte protoporphyrin, while still having a hemoglobin content above the threshold for anemia. Iron deficiency anemia is abnormal values of 2 out of 3 indicators with anemia (a hemoglobin content below the threshold for anemia). In the context of the present invention, ‘prevention’ of a disease or certain disorder also means ‘reduction of the risk’ of a disease or certain disorder and also means ‘treatment of a human subject at risk’ of said disease or said certain disorder.

In some embodiments, the composition is administered to a subject at a dose containing at least 1 g/day, preferably at least 3 g/day of the GOS+FOS mixture. In some embodiments, the composition is administered to a subject at a dose containing from 1 to 20 g/day, more preferably from 1 to 10 g/day, even more preferably from 1 to 5 g/day of the GOS+FOS mixture. In some embodiments, the composition is administered to a subject at a dose containing about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 g/day of the GOS+FOS mixture. In some embodiments, the composition is administered to a subject at a dose containing about 3 g/day or 7.5 g/day of the GOS+FOS mixture.

Preferably, the mixture of GOS+FOS is in a weight ratio of from 20:1 to 1:20, more preferably from 10:1 to 1:10, even more preferably from 9:1 to 1:9. In the most preferred embodiment, the mixture of GOS+FOS is in a weight ratio of 9:1. Preferably, the mixture of GOS+FOS is mixture of scGOS and lcFOS.

In some embodiments, the composition is administered to a subject at a dose of iron below 12.5 mg/day, more preferably below 10 mg/day, even more preferably below 5 mg/day. This level is below the levels of iron in common MNPs and it is considered to be unlikely to increase infectious morbidity. In some embodiments, the composition is administered to a subject at a dose containing from 1 to 12.5 mg/day, more preferably from 1 to 10 mg/day, even more preferably from 1 to 5 mg/day of iron. In some embodiments, the composition is administered to a subject at a dose of about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12 or 12.5 mg/day of iron. In a preferred embodiment, the composition is administered to a subject at a dose of about 3.6 mg/day of iron.

In some preferred embodiments, the composition comprises cereal, preferably wheat, with a mixture of GOS+FOS, preferably scGOS+IcGOS, in a ratio of 9:1 and iron, preferably ferrous fumarate, and is formulated to provide from 1 to 10 g, preferably 3 g or 7.5 g of the mixture of GOS+FOS per daily serving.

In some preferred embodiments, the composition is an instant formula comprising cereal, preferably wheat, ferrous fumarate (FeFum), preferably about 3.6 mg, and ascorbic acid (AA) in a 1:3 (Fe:AA) molar ratio, and about 7.5 g of a mixture of scGOS/lcFOS, per daily serving of 48 g dry product.

In some preferred embodiments, the composition is an instant milk formula comprising cereal, preferably wheat, ferrous fumarate (FeFum), preferably about 3.6 mg, and ascorbic acid (AA) in a 1:3 (Fe:AA) molar ratio, and about 7.5 g of a mixture of scGOS/lcFOS per daily serving of 48 g dry product.

In some preferred embodiments, the composition is an instant milk formula comprising cereal, preferably wheat, ferrous fumarate (FeFum), preferably about 3.6 mg, and ascorbic acid (AA) in a 1:3 (Fe:AA) molar ratio, and about 7.5 g of a mixture of scGOS/lcFOS in a 9:1 weight ratio (GOS:FOS) per daily serving of 48 g dry product.

In some preferred embodiments, the composition is an instant formula comprising cereal, preferably wheat, ferrous fumarate (FeFum), preferably 3.6 mg, and ascorbic acid (AA) in a 1:3 (Fe:AA) molar ratio, and about 3 g of a mixture of scGOS/lcFOS, per daily serving of 48 g dry product.

In some preferred embodiments, the composition is an instant milk formula comprising cereal, preferably wheat, ferrous fumarate (FeFum), preferably about 3.6 mg, and ascorbic acid (AA) in a 1:3 (Fe:AA) molar ratio, and about 3 g of a mixture of scGOS/lcFOS per daily serving of 48 g dry product.

In some preferred embodiments, the composition is an instant milk formula comprising cereal, preferably wheat, ferrous fumarate (FeFum), preferably about 3.6 mg, and ascorbic acid (AA) in a 1:3 (Fe:AA) molar ratio, and about 3 g of a mixture of scGOS/lcFOS in a 9:1 weight ratio (GOS:FOS) per daily serving of 48 g dry product.

The composition is preferably administered as a single dose per day. However, the composition can be administered to a subject in several portions, for example via two or three portions per day.

In an embodiment, the composition is administered to a subject all at once. In an embodiment, the composition is administered to a subject daily. In an embodiment, the composition is administered to a subject for at least one week, more preferably for two weeks, even more preferably for at least three weeks. In an embodiment, the composition is administered to a subject daily for at least one week, more preferably for two weeks, even more preferably for at least three weeks.

In some embodiments, the composition is administered to a subject to induce an acute effect, i.e. wherein a single administration of the composition improves the iron absorption in a subject.

In some embodiments, the composition is administered to a subject to induce a chronic effect, i.e. wherein the administration of the composition daily for at least one week, preferably two weeks, more preferably three weeks, improves the iron absorption in a subject.

In some embodiments, the composition is administered to a subject to induce a conditioning effect, i.e. wherein, following the administration of the composition, the iron absorption in a subject is improved without requiring the mixture of GOS+FOS. In other terms, the absorption of iron by the subject remains increased after the administration of the composition.

In some embodiments, the iron absorption (also “fractional iron absorption” or “FIA”) obtained with a composition of the invention is increased (or improved) by at least 10%, more preferably at least 20%, even more preferably at least 50% compared to the same composition devoid of the mixture GOS+FOS.

The iron absorption can be measured by techniques well known by one skilled in the art. Such techniques include, but are not limited to, measuring erythrocyte incorporation of a stable iron isotope into erythrocytes using an inductively coupled plasma mass spectrometer as shown in the Example.

The following Examples are put forth so as to provide those of ordinary skill in the art with a complete disclosure and description of how to make and use the present invention, and are not intended to limit the scope of what the Inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### DESCRIPTION OF THE FIGURES

FIG. 1. Study design and participant flowchart.

FIG. 2. Fractional iron absorption (FIA) from test meals consumed without and with GOS-FOS prebiotics before and after daily intake of GOS-FOS prebiotics for 3 weeks. (1) FIA from pooled 3.0 g/day and 7.5 g/day prebiotics doses; (2) FIA from 3.0 g/day and 7.5 g/day prebiotics doses separately.

### EXAMPLES

### Example I—Clinical Trial: GOS/FOS Impact on Iron Absorption in Presence of Iron of African Children

### Objectives

Providing a low dose of iron that is highly bioavailable and co-provision of prebiotics are promising strategies to mitigate the adverse effects of iron on the infant gut microbiota, and increase iron absorption. Thus, the goal of this study was to assess the effect of the prebiotics GOS and FOS on iron absorption, gut microbiome and inflammation from a wheat-based instant cereal newly formulated for complementary feeding during infancy in Sub-Saharan Africa. The primary objective was to measure fractional iron absorption (FIA) from a wheat-based instant cereal containing 3.6 mg ferrous fumarate (FeFum), with or without prebiotics (GOS+FOS).

### Study Outcomes

The primary outcome was FIA (measured by erythrocyte incorporation of stable iron isotopes) from the instant cereal containing FeFum, with and without GOS+FOS.

The secondary outcomes included: (1) the effects of acute vs. chronic consumption of GOS+FOS on FIA and (2) the dose-dependent GOS+FOS effect on FIA.

### Methodology

### Study Area

The study area was Msambweni and surrounding rural communities, Kwale County of southern coastal Kenya. The main economic activity in the area is subsistence farming with maize as the staple food crop. The typical local complementary food is “uji”, a liquid maize porridge.

### Study Population

### Inclusion Criteria

Age of 6-11 months at entry
General good health

### Exclusion Criteria

Hb<70 g/L.
Severe underweight (Z-score weight-for-age<-3).
Severe wasting (Z-score weight-for-height<-3).
Intake (>2 days) of iron-containing supplements or fortified foods in the past 2 months.
Antibiotic treatment in the past 4 weeks.

### Product Description and Composition

The products were instant milk based cereal products, designed for use in the weaning period containing the following ingredients:
Wheat flour (32.4%)
Skimmed milk powder (28.0%)
Product with 7.5 g prebiotics:
   Oligosaccharides (22.0% [scGOS (from milk) 20.4%, lcFOS 1.6%])
Product with 3 g prebiotics:
   Oligosaccharides (8.8% [scGOS (from milk) 8.2%, lcFOS 0.6%])
Whole milk powder (10.0%)
Palm oil
Emulsifier (soy lecithin)
Flavour (vanillin)
Sucrose (ingredient only in the recipe with 3 g prebiotics)
Vitamins: A, B1, B3, B5, B6, B8, B9, C, D3 and E
Minerals: Ferrous fumarate (iron source)

The product was provided as powder to be mixed with water. The recommended daily intake for children of 6 months or older was 48 grams to be mixed with 180 ml water. In the clinical trial setting, a product preparation kit was supplied, which included a measuring cup, scoop and spatula for preparation of the cereals.

The nutritional compositions of instant milk based cereals with 7.5 or 3 grams of scGOS/lcFOS are given in Tables 1 and 2 respectively.

TABLE 1
Nutritional composition of instant milk based cereals with 7.5 grams of
scGOS/lcFOS per daily serving (48 grams dry product).

| | | Amounts | Amounts of dry | Amounts per daily |
|---|---|---|---|---|
| | | per 100 g | product per 100 | portion prepared |
| 7.5 grams scGOS/IcFOS | | of dry product | kcal | product** |
| Components | | Target value* | Target value* | Target value* |
| Energy | kJ | 1605 | — | 771 |
| | kcal | 383 | — | 184 |
| Macronutrients | | | | |
| Proteins | g | 16.6 | 4.3 | 8.0 |
| Carbohydrates | g | 44.0 | 11.5 | 21.1 |
| Of which sugars^{#} | g | 24.8 | 6.5 | 11.9 |
| Fats | g | 11.4 | 3.0 | 5.5 |
| Of which saturated fatty acids | g | 6.0 | 1.6 | 2.9 |
| (Dietary) fibre^{#} | g | 19.2 | 5.0 | 9.2 |
| Micronutrients | | | | |
| Iron (Fe) | mg | 7.3 | 1.9 | 3.5 |
| Calcium | mg | 526 | 123 | 227 |
| Sodium (Na) | mg | 159 | 41 | 76 |
| Vitamin A | μg | 525 | 137 | 252 |
| Thiamin (vitamin B1) | mg | 0.5 | 0.1 | 0.2 |
| Niacin (vitamin B3) equivalent | mg | 4.5 | 1.2 | 2.3 |
| Pantothenic acid (vitamin B5) | mg | 1.9 | 0.5 | 0.9 |
| Vitamin B6 | mg | 0.3 | 0.1 | 0.2 |
| Biotin (B8) | μg | 25 | 6.6 | 12 |
| Folic acid (B9) | μg | 48 | 13 | 23 |
| Vitamin C | mg | 68 | 18 | 33 |
| Vitamin D3 | μg | 6.0 | 1.6 | 2.9 |
| Vitamin E | mg | 2.8 | 0.7 | 1.3 |
| Other | | | | |
| Salt | g | 0.40 | 0.1 | 0.2 |

*tolerable ranges: vit A and D3: −20% +40%, vit C: −20% +100%, other vitamins: −20% +80%, proteins and carbs: −15% +15%, sugars and dietary fibre: −20% +20%, fat: −15% +15%, saturated fatty acids: −30% +30%, Ca and Na: −20% +30%, iron: −20% +20%);
**a daily portion consists of 48 g product and 180 ml water = 228 RTF;
^{#}Prebiotics are added in forms of carbohydrates (sugars) and dietary fibre.

TABLE 2
Nutritional composition of instant milk based cereals with 3
grams of scGOS/lcFOS per daily serving (48 grams dry product).

| | | | Amounts of dry | Amounts per |
|---|---|---|---|---|
| | | | product per 100 | daily portion |
| | | Amounts per 100 | kcal | prepared |
| 3 grams scGOS/IcFOS | | g of dry product | Target | product** |
| Components | | Target value* | value* | Target value* |
| Energy | kJ | 1659 | — | 796 |
| | kcal | 392 | — | 188 |
| Macronutrients | | | | |
| Proteins | g | 17.8 | 4.54 | 8.5 |
| Carbohydrates | g | 61.0 | 15.6 | 29.3 |
| Of which sugars^{#} | g | 30.9 | 7.9 | 14.8 |
| Fats | g | 6.3 | 1.6 | 3.0 |
| Of which saturated fatty acids | g | 3.2 | 0.8 | 1.5 |
| (Dietary) fibre^{#} | g | 9.8 | 2.5 | 4.7 |
| Micronutrients | | | | |
| Iron (Fe) | mg | 7.5 | 1.9 | 3.6 |
| Calcium | mg | 478 | 122 | 230 |
| Sodium (Na) | mg | 160 | 41 | 77 |
| Vitamin A | μg | 396 | 101 | 190 |
| Thiamin (vitamin B1) | mg | 0.6 | 0.2 | 0.3 |
| Niacin (vitamin B3) equivalent | mg | 4.7 | 1.2 | 2.5 |
| Pantothenic acid (vitamin B5) | mg | 2.0 | 0.5 | 1.0 |
| Vitamin B6 | mg | 0.4 | 0.1 | 0.2 |
| Biotin (B8) | μg | 26 | 6.6 | 12 |
| Folic acid (B9) | μg | 50 | 13 | 24 |
| Vitamin C | mg | 69 | 18 | 33 |
| Vitamin D3 | μg | 5.6 | 1.4 | 2.7 |
| Vitamin E | mg | 2.5 | 0.6 | 1.2 |
| Other | | | | |
| Salt | g | 0.4 | 0.1 | 0.2 |

*tolerable ranges: vit A and D3: −20% +40%, vit C: −20% +100%, other vitamins: −20% +80%, proteins and carbs: −15% +15%, sugars and dietary fibre: −20% +20%, fat: −15% +15%, saturated fatty acids: −30% +30%, Ca and Na: −20% +30%, iron: −20% +20%);
**a daily portion consists of 48 g product and 180 ml water = 228 RTF;
^{#}Prebiotics are added in forms of carbohydrates (sugars) and dietary fibre.

### Study Design

This study had three arms, each with 65 infants (total n=195), who were randomized to one of the three groups at enrolment. Through another randomization, a subset of infants (n=70) from groups 1 and 2 were enrolled in a stable iron isotope absorption study. During screening/recruitment (visit 1), for the inclusion criteria, hemoglobin (Hb) concentration from a finger prick and anthropometrics (height, weight, mid-upper arm and head circumference) were measured; demographics, medical history and feeding habits were assessed using a questionnaire. Written informed consent was obtained from the families of the infants, and ethics committees approved the protocol in Switzerland and Kenya.

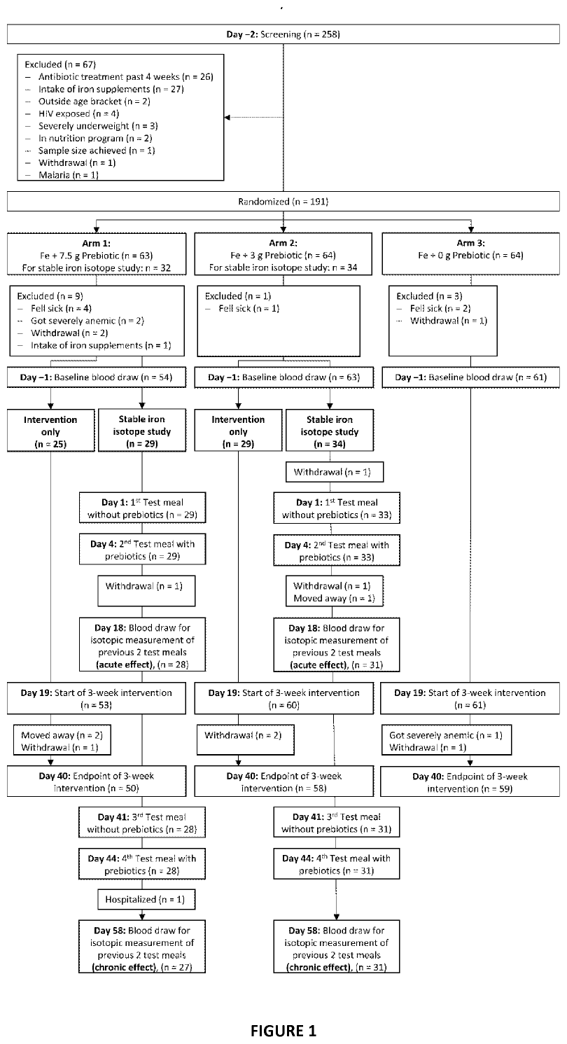

The overall study design is shown in FIG. 1. Infants in all three groups consumed daily, for 3 weeks, the wheat-based instant cereal containing 3.6 mg iron as FeFum.
Group 1 consumed the cereal with a 7.5 g dose of GOS+FOS, 9:1
Group 2 consumed the cereal with a 3 g dose of prebiotic GOS+FOS, 9:1.
Group 3 consumed the cereal without a prebiotic.

Distribution of the wheat-based instant cereal, compliance and monitoring of infant health was done weekly.

A subset of infants in groups 1 and 2 (n=35 in each group; total n=70) were randomly selected to participate in the stable iron isotope absorption study. Using G*Power Statistical Program v.3.1.3, it was calculated the sample size necessary to detect a 42% difference in FIA between the two arms and a 30% difference in FIA within the infants (without versus with prebiotics, and acute versus chronic effect) based on a standard deviation (SD) of 0.228 from log-transformed FIA from previous studies by our laboratory, and assuming a type I error rate of 5% and power of 80%. The sample size calculation indicated that 29 infants were needed in each arm for the stable iron isotope study. Anticipating a drop-out rate of 18%, we aimed to enroll 35 infants per group.

Each infant consumed four test meals of labeled wheat-based instant cereal containing ⁵⁷Fe- and ⁵⁸Fe-labeled FeFum with and without the prebiotics. Two of the labeled test meals were fed 2 weeks before beginning the 3-week intervention study, and two of the labeled test meals were fed at the end of the 3-week intervention study. At baseline, a 2 mL blood sample was collected from the infants. Additionally, the child's health, body height and weight, demographic information, medical history and feeding habits using a short questionnaire were measured. Additionally, the child's health, body height and weight, mid-upper arm and head circumference, demographic information, medical history and feeding habits using a short questionnaire were measured. The infants then consumed two test meals on two mornings separated by two days (days 1 and 4):
For group 1, the first test meal contained 3.6 mg iron as ⁵⁷FeFum without GOS+FOS; the second contained 1.5 mg iron as ⁵⁸FeFum and 2.09 mg iron as ⁵⁶Fe and 7.5 g of GOS+FOS.
For group 2, the first test meal contained 3.6 mg iron as ⁵⁷FeFum without GOS+FOS; the second contained 1.5 mg iron as ⁵⁸FeFum and 2.09 mg iron as ⁵⁶Fe and 3 g of GOS+FOS.

Fourteen days after the second meal, a 2 mL venipuncture blood sample was collected for analysis of iron incorporation of the stable iron isotopes into erythrocytes. Additionally, the child's height and weight were measured. Infants in the absorption sub-study then rejoined the three groups described above in the 3-week intervention study. Every week, instant cereal was distributed in a box with a measuring cup and a feeding cup, which was provided by the study team; compliance and monitoring of infant health was done. After 3 weeks of feeding of the cereal at home, the subset of infants in the stable isotope absorption study consumed two labeled test meals, on two mornings separated by two days.
For group 1, the third test meal contained 3.6 mg iron as ⁵⁷FeFum without GOS+FOS; the fourth contained 1.5 mg iron as ⁵⁸FeFum and 2.09 mg iron as ⁵⁶Fe and 7.5 g of GOS+FOS.
For group 2, the third test meal contained 3.6 mg iron as ⁵⁷FeFum without GOS+FOS; the fourth contained 1.5 mg iron as ⁵⁸FeFum and 2.09 mg iron as ⁵⁶Fe and 3 g of GOS+FOS.

Fourteen days after the fourth test meal, a 2 mL venipuncture blood sample was collected for analysis of iron incorporation of the stable iron isotopes into red blood cells (RBCs). Additionally, the child's height and weight were measured.

### Laboratory Analyses

The ⁵⁷FeFum and ⁵⁸FeFum were prepared by Dr. Paul Lohmann GmbH from ⁵⁷Fe- and ⁵⁸Fe-enriched elemental iron (95.78% and 99.9% isotopic enrichment). The labeled iron compounds were analyzed for iron isotopic composition and the tracer iron concentration was analyzed via inverse isotope-dilution mass spectrometry. Fractional iron absorption (FIA) was determined by measuring erythrocyte incorporation of the stable iron isotope into erythrocytes. Whole blood samples were mineralized in duplicate by microwave-assisted digestion in nitric acid, followed by iron separation. Iron isotope ratios were measured using an inductively coupled plasma mass spectrometer equipped with a multi-collector system for simultaneous iron beam detection. The amount of ⁵⁷Fe and ⁵⁸Fe isotopic labels in blood 14 days after the administration of the second and fourth test meal, was calculated based on the shift in iron-isotopic ratios and the estimated amount of iron circulating in the body. Circulating iron was calculated based on Hb concentrations and blood volume. The calculations were based on the methods described by Turnlund et al. (Anal. Chem., 65(13):1717-22, 1993) and Cercamondi et al. (J. Nutr., 143(8):1233-9, 2013) considering that iron isotopic labels were not monoisotopic. For the calculation of FIA, we assumed a 75% incorporation of the absorbed iron (Tondeur et al., Am. J. Clin. Nutr., 80(5):1436-44, 2004). In plasma, iron status markers (plasma ferritin [PF] and soluble transferrin receptor [sTfR]), systemic inflammation markers (C-reactive protein [CRP] and α-1-glycoprotein [AGP]) and retinol binding protein (RBP) were analyzed using a multiplex immunoassay. Expected CRP and AGP concentrations for healthy infants are <5 mg/L and <1 g/L, respectively. PF was adjusted for inflammation using BRINDA correction. Anemia was defined as Hb<110 g/L; iron deficiency (ID) was defined as PF<12 μg/L and/or sTfR>8.3 mg/L, and iron deficiency anemia (IDA) as ID and anemia. Z-scores for weight-for-age, weight-for-length and length-for-age were calculated using WHO Anthro software v.3.2.2.

### Statistical Analysis

Values in the text and in tables are presented as means±SD for normally distributed data, and as medians (25th-75th percentiles) for non-normally distributed data. When data were not normally distributed, transformation was performed before statistical analysis. Person's Chi-squared tests were used to compare categorical variables between groups at baseline, and where the sample size was not sufficient, Fisher's exact tests were used. Independent-samples t-tests were used to compare continuous variables between the 2 groups at baseline. For FIA, paired sample t-tests were used for normally distributed data and related samples Wilcoxon signed rank tests were used for not normally distributed data. The Bonferroni adjustment was used to correct the results for multiple comparisons (level of significance: p<0.017). Linear mixed effect model analyses were used to assess whether the prebiotic dose affects the 1) acute prebiotic effect (dependent variable: FIA before intervention without and with prebiotic; fixed effects: prebiotic (without/with), dose (7.5 g/3.0 g)); 2) chronic prebiotic effect (dependent variable: FIA without prebiotic before and after intervention; fixed effects: time (before intervention/after intervention, dose (7.5 g/3.0 g)); 3) combined acute and chronic prebiotic effect (dependent variable: FIA before intervention without prebiotic and after intervention with prebiotic; fixed effects: acute+chronic prebiotic (yes/no), dose (7.5 g/3.0 g)). Serum ferritin and C-reactive protein measured at the time of the absorption studies were added as covariates to these models.

### Results

312 infants were screened for eligibility at Day 2 (FIG. 1). A total of 195 infants were eligible and randomly assigned to one of the three study arms (65 infants per arm). In arms 1 and 2, 35 of the 65 infants were each randomly assigned to participate in the stable iron isotope study. Subsequently, 29 infants in arm 1, and 33 infants in arm 2, consumed the first two test meals, and 28 and 31 infants, respectively, gave blood 2 weeks later for the isotopic measurements. All infants from the stable iron isotope study underwent the 3-week intervention and had their intervention endpoint. Then, 28 infants in arm 1, and 31 infants in arm 2 consumed the last two test meals. One infant in arm 1 was hospitalized before the final blood draw and was excluded.

FIA values from n=5 infants pre-intervention and from n=5 infants after intervention who had a CRP>5 mg/L at the time of the absorption studies, indicating an acute infection, were excluded. One FIA value from one child in arm 1 who vomited immediately after the test meal administration was further excluded. Therefore, to determine the acute and chronic effect of prebiotics on FIA, in arm 1, 25 infants, and in arm 2, 28 infants finished the stable iron isotope study and were included into the analyses. Age, anthropometric measurements, hemoglobin, anemia, iron and inflammation status in all of the enrolled Kenyan infants at baseline are shown in Table 3.

TABLE 3
Age, anthropometric measurements, iron status, inflammation
and vitamin A status in Kenyan infants at baseline.

| | Arm 1 | Arm 2 | |
|---|---|---|---|
| | Fe + 7.5 g Prebiotic | Fe + 3 g Prebiotic | P |
| n | 29 | 34 | |
| Female/Male, n (%) | 14 (48.3)/15 (51.7) | 17 (50.0)/17 (50.0) | 0.891 |
| Age, mo¹ | 7.8 ± 1.3 | 8.0 ± 1.3 | 0.594 |
| Anthropometrics | | | |
| Body length, cm | 68.3 ± 3.0 | 68.8 ± 3.0 | 0.450 |
| Body weight, kg | 7.9 ± 1.3 | 7.9 ± 1.1 | 0.826 |
| Weight-for-length Z-score | −0.11 ± 1.44 | −0.35 ± 1.11 | 0.453 |
| Weight-for-age Z-score | −0.41 ± 1.33 | −0.51 ± 0.97 | 0.730 |
| Length-for-age Z-score | −0.49 ± 1.07 | −0.35 ± 0.76 | 0.532 |
| Iron status | | | |
| Hemoglobin, g/L² | 103 (98-108) | 106 (100-115) | 0.457 |
| Anemia, n (%)³ | 24 (82.6) | 23 (67.6) | 0.170 |
| Plasma ferritin, μg/L | 7.2 (3.1-16.6) | 14.2 (8.5-23.4)⁴ | 0.018 |
| <12 μg/L, n (%) | 19 (65.5) | 13 (39.4)⁴ | 0.040 |
| Plasma ferritin adjusted, μg/L⁵ | 6.4 (2.9-12.5) | 11.0 (8.1-19.0)⁴ | 0.020 |
| <12 μg/L, n (%) | 21 (72.4) | 19 (57.6)⁴ | 0.223 |
| Soluble transferrin receptor, mg/L | 11.0 (8.9-16.2) | 11.0 (9.5-16.1)⁴ | 0.570 |
| >8.3 mg/L, n (%) | 23 (79.3) | 26 (78.8)⁴ | 0.960 |
| Iron deficiency, n (%)⁶ | 23 (79.3) | 27 (81.8)⁴ | 0.803 |
| Iron deficiency anemia, n (%)⁷ | 22 (75.9) | 19 (57.6)⁴ | 0.153 |
| Systemic inflammation | | | |
| C-reactive protein, mg/L | 0.15 (0.03-1.14) | 0.24 (0.02-2.41)⁴ | 0.965 |
| 0.05-4.99 mg/L, n (%) | 16 (55.2) | 17 (51.5)⁴ | 0.773 |
| ≥5 mg/L, n (%) | 3 (10.3) | 3 (9.1)⁴ | 0.868 |
| α-1-glycoprotein, g/L | 0.63 (0.49-0.77) | 0.67 (0.45-0.91)⁴ | 0.520 |
| ≥1 g/L, n (%) | 3 (10.3) | 7 (21.2)⁴ | 0.312 |
| Inflammation, n (%)⁸ | 4 (13.8) | 7 (21.2)⁴ | 0.445 |

¹Mean ± SD all such values.
²Median (25th-75th percentiles) all such values.
³<110 g/L.
⁴n = 33.
⁵Adjusted for inflammation using BRINDA correction.
⁶Adjusted plasma ferritin <12 μg/L and/or transferrin receptor >8.3 mg/L.
⁷Anemia and iron deficiency.
⁸CRP ≥5 mg/L and/or AGP ≥1 g/L.

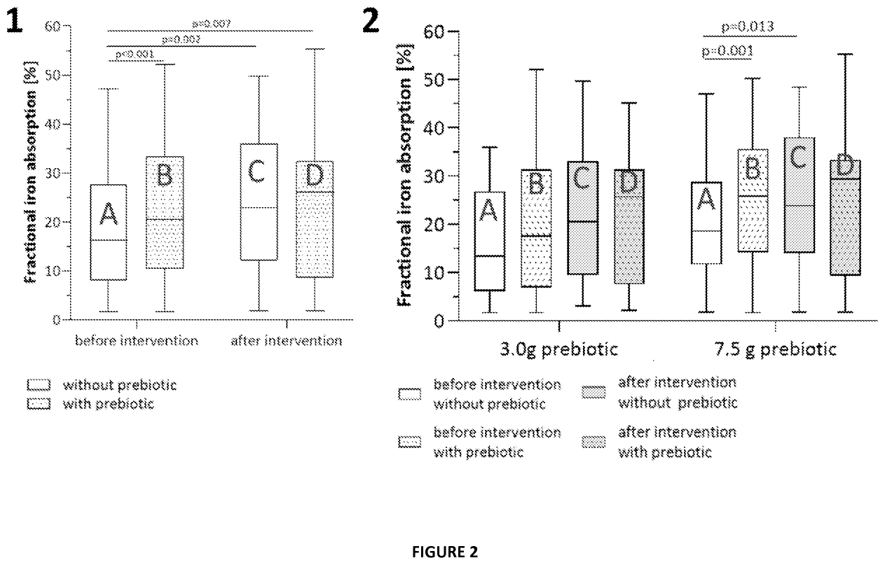

FIA from test meals consumed without and with prebiotics before and after intervention are shown in Table 4 and FIG. 2.

In a pooled analysis assessing FIA from both prebiotic doses (Table 4 and FIG. 2):
the addition of prebiotics to the test meal at baseline resulted in a higher FIA compared to the test meal without prebiotics (p<0.001)
the intake of prebiotics daily for 3 weeks resulted in a higher FIA from the test meal without prebiotics after intervention compared to the test meal without prebiotics before intervention (p=0.002)
FIA from the test meal consumed with prebiotics after intervention was higher compared to FIA from the test meal without prebiotics before intervention (p=0.007)

TABLE 4
Fractional iron absorption (FIA) from test meals consumed without and with
prebiotics before and after daily intake of prebiotics for 3 weeks.

| | Pooled prebiotic doses | | 3.0 g prebiotic | | 7.5 g prebiotic | |
|---|---|---|---|---|---|---|
| | Without | With | Without | With | Without | With |
| | prebiotic | prebiotic | prebiotic | prebiotic | prebiotic | prebiotic |
| FIA before | 16.3 | 20.5 | 13.4 | 17.6 | 18.6 | 25.8 |
| intervention, | (8.0-27.6) | (10.4-33.4) | (6.1-26.8) | (6.9-31.4) | (11.7-28.7) | (14.2-35.6) |
| % | (n = 54) | (n = 53) | (n = 28) | (n = 28) | (n = 26) | (n = 25) |
| FIA after | 22.9 | 26.0 | 20.5 | 25.7 | 23.9 | 29.4 |
| intervention, | (12.2-36.1) | (8.5-32.4) | (9.5-33.1) | (7.6-31.4) | (14.0-38.1) | (9.3-33.4) |
| % | (n = 53) | (n = 53) | (n = 28) | (n = 28) | (n = 25) | (n = 25) |

In an analysis assessing FIA from the 7.5 g prebiotic dose (Table 4 and FIG. 2):
the addition of 7.5 g prebiotics to the test meal at baseline resulted in a higher FIA compared to the test meal without prebiotics (p=0.001)
the intake of 7.5 g prebiotics daily for 3 weeks resulted in a higher FIA from the test meal without prebiotics after intervention compared to the test meal without prebiotics before intervention (p=0.013)
there was a trend for a higher FIA from the test meal consumed with 7.5 g prebiotics after intervention compared to FIA from the test meal without prebiotics before intervention (p=0.102)

In an analysis assessing FIA from the 3.0 g prebiotic dose (Table 4 and FIG. 2):
the addition of 3.0 g prebiotics to the test meal at baseline shows a trend for a higher FIA compared to the test meal without prebiotics (p=0.094)
the intake of 3.0 g prebiotics daily for 3 weeks shows a trend for a higher FIA from the test meal without prebiotics after intervention compared to the test meal without prebiotics before intervention (p=0.034)
there was a trend for a higher FIA from the test meal consumed with 3.0 g prebiotics after intervention compared to FIA from the test meal without prebiotics before intervention (p=0.075)

Linear mixed effect model analysis showed no effect of the prebiotic dose on FIA; specifically, 1) the addition of prebiotics before intervention (p=0.990); 2) the intervention (p=0.625); and 3) the addition of prebiotics after intervention (p=0.826).

### INTERPRETATION AND CONCLUSIONS

1. The 26% increase in FIA from A to B represents the enhancing effect of a single dose of prebiotics given with the iron.

2. The 41% increase in FIA from A to C represents the conditioning effect of 3 weeks of prebiotics.

3. The 60% increase from A to D represents the effect of a dose of GOS+FOS given with the iron and 3 weeks of conditioning.

The increase in 3 above probably best represents the benefit of adding GOS+FOS to the wheat-based cereal with FeFum: a 60% increase in absorption between the iron-fortified cereal without GOS+FOS and the iron fortified cereal with GOS+FOS when consumed every day, as in ‘real life’.

For the primary outcome:
The addition of the GOS+FOS results in a significant increase in FIA from the wheat-based cereal containing FeFum.

For the secondary outcomes:
The addition of the GOS+FOS results in a significant increase in both acute and chronic FIA from the wheat-based cereal containing FeFum.
In the paired t-tests on the above outcomes by dose, the 7.5 g dose has a more statistically significant effect than the 3.0 g dose; however, the mixed model analysis shows no effect of the prebiotic dose on FIA.

1. A composition comprising at least a mixture of galacto-oligosaccharides and fructo-oligosaccharides (GOS+FOS), cereal and iron.

2. The composition according to claim 1, wherein the mixture of GOS+FOS is in a weight ratio from 20:1 to 1:20.

3. The composition according to claim 1, wherein the GOS are short chain GOS (scGOS) and/or the FOS are long chain FOS (lcFOS).

4. The composition according to claim 1, wherein the cereal is selected from the group consisting of wheat, millet, sorghum, rice, teff, rye, spelt, barley, oat, soy, maize, semolina, buckwheat, tapioca, quinoa, amaranth, brown rice, wild rice, bulgur, farro, freekeh, khorasan wheat and combinations thereof.

5. The composition according to claim 1, wherein the cereal is non-fermented.

6. The composition according to claim 1, wherein the composition comprises from 1 to 10 mg iron per 100 g dry weight of the total product.

7. The composition according to claim 1, wherein the iron is ferrous fumarate.

8. The composition according to claim 1, wherein the composition further comprises a food grade liquid selected from water, follow on formula or milk.

9. The composition according to claim 1, wherein said composition is a nutritional composition, a nutraceutical composition, a nutritional supplement and/or a food composition.

10. (canceled)

11. A method for treating or preventing anemia and/or iron deficiency in a subject comprising administering the composition according to claim 1.

12. A method for increasing iron absorption, iron bioaccessibility and/or iron bioavailability in a subject comprising administering the composition according to claim 1.

13. The method according to claim 11, wherein the subject is selected from the group consisting of infants, young children, women of reproductive age including pregnant or lactating women and elderly.

14. A method for improving cognitive development, improving motor development and/or improving socio-emotional development in a human subject with an age of 0 to 36 months or for preventing cognitive disorders, motor disorders and/or socio-emotional disorders in a human subject with an age of 0 to 36 months comprising administering the composition according to claim 1.

15. The method use according to claim 11, wherein the composition is administered to the subject daily.

16. The method according to claim 11, wherein the composition is administered to the subject at a dose containing from 1 to 20 g/day of the GOS+FOS mixture.

17. The method according to claim 11, wherein the composition is administered to a subject at a dose containing from 1 to 12.5 mg/day of iron.

18. The method according to claim 11, wherein the composition is administered to the subject for at least one week.

19. The method according to claim 11, wherein a single administration of the composition improves the iron absorption in the subject.

20. The method according to claim 11, wherein the administration of the composition daily for at least one week improves the iron absorption in the subject.

21. The method according to claim 11, wherein, following the administration of the composition, the iron absorption in the subject is improved without requiring the mixture of GOS+FOS.
